(19) United States
(12) Patent Application Publication
(10) Pub. No.: US 2002/0150551 A1
(43) Pub. Date: Oct. 17, 2002

(54) GRADUAL MODIFICATION, SUPER-AGONISTS AND ANTAGONISTS OF SIGNAL - PROTEINS AND PEPTIDES

(76) Inventors: VICTOR SMIT, DELFT (NL); WILLEM HUPPES, HILVERSUM (NL)

Correspondence Address: FITCH EVEN TABIN AND FLANNERY, 120 SOUTH LA SALLE STREET, SUITE 1600, CHICAGO, IL, 606033406

(21) Appl. No.: 08/807,506

(22) PCT Filed: Aug. 30, 1995
(86) PCT No.: PCT/NL95/00292

(30) Foreign Application Priority Data
Aug. 31, 1994 (NL) 9401404
May 10, 1995 (NL) 1000332

(51) Int. Cl. A61K 45/00

(52) U.S. Cl. 424/85.1

(57) ABSTRACT

Present invention concerns the specific gradual modification of signal proteins and peptides. By a combination of modification with the localization thereof with protease treatment and new forms of mass spectrometry, a very specific modification of the protein or peptide can be achieved. This enables the introduction of a desired change in biological activity, preferably an enhanced activity, an antagonistic activity and/or a cell inhibitory activity. The antagonistic or cell inhibitory activity could be realized by chemical modification, namely an alkylation with specificity for His residues that were localized in or near a catalytic and/or Zinc binding center of human Interleukin-3. One that is skilled in the art can also easily use this invention to generate inhibitors and/or antagonists, preferably by molecular biological modifications, chemical modifications and/or alkylations, preferably by lodo-Acetate. Preferably these modifications are directed against His residues, other catalytic residues and/or Zinc binding residues. From additional results and data it can be deduced that the invention can also be applied easily on other signal substances. The results can also be well accomplished by DNA-constructs which also includes gene-therapy. Thus the presented methods, obtained substances and the use thereof are embodied in the invention.

### SCIENTIFIC FIELD OF THE INVENTION

This invention is on the area of the chemical modification technology of biologically active proteins and peptides. More specifically it concerns use of chemical modification to obtain a protein or peptide with superior properties or with new or even counteracting properties. In addition, the invention also concerns a new method for structure-function analysis by using gradual chemical modification, a biological principle, namely the catalytic activity of signal peptides and the successful abrogation thereof, leading to a very effective inhibitor of Acute Myeloid Leukemia cells.

As illustration, but not as limitation, we show the chemical modification of human IL-3, a protein that also has a substantial therapeutic value after modification. This patent- description also contains specific examples within the therapeutic field of the invention.

### FIELD OF THE APPLICAATION

There are two possible fields of applications that are important for the invention: A field for an IL-3 with superior properties (Superagonist) or a field for an IL-3 with counteracting or new properties (Antagonist). In this patent description Superagonists are for instance an IL-3 with a lowered antigenecity and/or a higher biological activity and/or a higher stability.

Possible applications of IL-3 Superagonists are:

reduction of the cytopenic phase after myelo-ablative therapy like after inductive therapy for bone marrow transplantation or after accidental radiation.

induction of a synchronized cell-cycle of cells with an IL-3 receptor, for instance for chemotherapy of leukemia's.

induction of enhancement of the IL-3-dependent progeny both for the number of cells and their activation, for treatment of diseases like worm infections, tuberculosis, fungal infections and certain viral infections.

selective outgrowth of the bone marrow towards nucleus containing cells except lymphocytes, for instance with burning wounds and non-homologous skin- transplants.

Some, but not all, examples of applications of signal-substance antagonists (with counteracting or cell-inhibitory activity), more specifically of EL-3 are:

inhibition and/or neutralization of myeloid cells in bone marrow transplantation.

myelo-suppression in auto immune-diseases. cancer and diseases of the blood forming organs, like sickle cell anemia and thallisemia. treatments to cure all sorts of cancer that involve cells with the IL-3 receptor. more specifically almost all forms of acute myeloid leukemia or chronic myeloid leukemia, B-cell lymphoid tumors or other forms of cancer that are stimulated by IL-3, for instance certain follicle cell tumors.

induction of self-tolerance towards the tissues in auto immune diseases like arthritis, rheumatic arthritis and diseases of the central nervous system, by suppression or elimination of lymphoid cells that contain the IL-3 receptor. This can also lead to the impaired generation and elimination of effector cells like the eosinophilic granulocyte. Finally it is possible that there is a direct interaction with these cells, thus enabling a direct cure of the eosinophilic syndrome. This is also of great importance in acute phases of worm infections and hypersensitivity reactions to for instance medicinal drugs.

curing eosinophilic syndromes like eosinophilic gastritis and enteritis, fascitis, granulomatosis, sinusitis, pneumonia, asthma, Churg Strauss syndrome and other angitis treatments of the shock syndrome for instance by killing or suppressing the number of effector cells.

ablation or suppression of cells with an IL-3 receptor, like lymphoid cells and/or effector cells like the eosinophilic granulocyte, for the treatment of allergies. In these cases both suppression of the allergy and induction of tolerance towards the antigen are possible.

other allergic reactions where the action of EL-3 is involved.

treatments to prevent metastases that are stimulated by IL-3 mediated adhesion.

treatment of infectious diseases, for instance by suppressing an acute phase where there is an occurrence of excessive amounts of growth factor in the blood stream.

Treatment of HIV-infection and/or AIDS by suppression of B-cells and B-cell antibody production (that protect the HIV-virus against the cellular resistance of the host).

One or more of these applications can also be mentioned for other growth factors like the other Interleukins 1-8 , GM-CSF, TNF and gamma IFN, which are also good candidates for modification according to this invention. Hereby ‘IL-3’ is to be replaced by the other signal substance. Since the modes of interaction of the various substances in the various diseases can differ from substance to substance, several synergistic effects can also be expected. Therefore these are also ebodied in the invention. Finally, the following applications can be added or specified:

IL-1 inhibition to suppress IL-1 stimulated metastases of melanoma's and forms of lung cancer.

IL- I inhibition to suppress Alzheimer's disease

IL-2 inhibition to suppress capillary leak syndrome

IL-2 inhibition to suppress periodontitis

IL-4 inhibition to suppress periodontitis

IL-4 inhibition to suppress ILAo stimulated viruses like for instance the radiation leukemia virus in mice.

IL-5 inhibition to suppress IL-5 stimulated respiratory tract infections.

IL-6 inhibition to suppress Rheumatoid arthritis.

IL-10 inhibition to suppress infections of Mycobacterium.

With an in vivo example it is shown that there is a possibility to effectively suppress an AIDS-virus infection. This suppression is based on the reduction of the antibody-level that can be achieved by the suppression of antibody-producing B-cells. The fact that this can lead to an effective suppression of AIDS can be attributed to the following causes: (1) The HIV-virus infects the hystiocytic cells (macrophage like cells) preferably by opsonisation with antibodies. Macrophage tropism and the necessity to of infection of infection of these histiocytic cells for persisting infection is described in AIDS Res. and Human Retroviruses 9: 669 (1993) and references. By the reduction of the antibody- levels this form of infection can be suppressed. (2) Antibodies can protect HIV and/or HIV-infected cells against cellular immunity. This explains the defective overall resistance in the asymtomatic phase of the HIV infection in spite of very high levels of in vitro neutralizing antibodies and a demonstrated cellular resistance. As demonstrated in the example the lowering of the antibody-levels can result in an effective cellular immunity against the virus and the virus infected cells and even the elimination of the virus. Therefore, the inhibition of B-cells with for instance antagonists can lead to a cure of the HIV infection.

Since it is perceptible that the modified signal peptides can also be generated by molecular biology, these mutant proteins, DNA-constructs and the use thereof are also considered to be within the scope of the invention. Hereby, one can include :ene-therapy with the constructs that contain the code for such peptides and/or proteins. The use in gene- therapy can result in cells that are producing and excreting the super-agonist or the antagonist. Therefore, in one of the examples there is also an elaboration on the possibility to construct a growth factor with a reduced stability. This can be used especially in combination with antagonistic action, thus resulting in a selective administration whereby the high rate of breakdown of the antagonist can contain the action to a very localized environment. This is particularly interesting in gene-therapy for solid tumors. If there is such a cell in or near the tumor, especially this tumor will experience the exposure to the maximal effect. If there is an additional instability of the produced signal peptide, the effect of the peptide can be very localized, thus leading to the expectation of fewer side-reactions. In one of the examples it is demonstrated that it is possible to generate a signal peptide with a lowered stability. Since such peptides can also be generated with deletion and/or substitution mutants, such gene-therapeutic applications and gene therapy- constructs are also considered to be within the field of applications of the invention.

This patent-description also elaborates on the use of chemical modification, protease treatment and mass spectrometry. This can be applied to every modification of every peptide or protein, provided it can be specifically fragmented for instance with protease's. The quantitative structure-function analysis part of the description contains the successful use of the combination of gradual chemical modification, protease treatment and laser desorption mass spectrometry with biological assays. Therefore, the application of this analysis on any peptide and/or protein that can be modified, specifically fragmented and is biologically active is also considered to be within the scope of the invention. It is also possible to use other mass spectrometric techniques like electrospray mass spectrometry, which is particularly suitable after for instance exoprotease treatment. Finally, Be description of the invention also discloses the importance of metal ions with more than I valence in charge, by preference Zinc-ions, especially for catalytic activity and the subsequent efficacy. Therefore, it is possible to influence the growth factor action by manipulating the (local) metal ions concentrations. Since there is a usual optimum in efficacy of the metal ions concentration, and any concentration below or beyond this optimum can lead to reduction of efficacy of the growth factor. This enables manipulation of the efficacy of the growth factor. In this way also indirect therapeutic effects can be achieved with metal-, preferably Zinc ions. This can be used in treatment of skin-diseases like wrath's in the form of ointments. Also, the use of inhalation-sprays for treatment of lung-affects is possible.

### BACKGROUND OF THE INVENTION

Human Interleukin-3

Interleukin-3, which was first isolated from T-cells as a glycoprotein, has been demonstrated to act on bone-marrow cells. Either with or without other growth factors it has been shown to induce the formation of various blood cells from these bone-marrow cells. Human IL-3 has been cloned for the first time in 1987 by Dorssers et al. who used a humar. c-DNA library and hybridization with a probe of mouse DNA ( Gene 55: 115 (1987).

Structure-function relationship of human Interleukin-3: Several articles have been published concerning the structure-function relationship of human Interleukin-3 (J. Biol. Chem. 266: 21310 (1991); J. Clin. Invest 90: 1879(1992); J. Immunol. 146: 83 (1991); EMBO J. 10:2125 (1991); Proc. Natl. Acad. Sci. USA 89: 11842 (1992)). In their research they all used molecular biological techniques like the generation of deletion and substitution mutants. In the case that substitution mutants were used, the choice was based on the amino-acid homology ofIL-3 with other species (mouse, rhesus monkey, gibbon) or it was based on deliberate changes of polarity or structure. With deletion mutants the protein was scanned for biological importance by removal of parts of the protein. Because of practical problems with regard to purification of the mutant- proteins(muteins) these mutant proteins have never been checked for major structural changes. This is a major problem since these structural changes usually do occur. As described, sometimes they are even deliberately introduced. As a result any statement regarding the involvement of various amino acids on the biological activity can only be made with the greatest reservations.

Present invention has a different approach. The native IL-3 is used as starting material. By using stepwise gradually increasing modification an extra selectivity is introduced in the chemical reaction. The modification that occurs simultaneous with the first change in biological activity can be simply localized with specific protease's and new forms of mass spectrometry. As a result, important amino acid residues can be quickly localized and minimal change ( and therefore maximal control ) can be achieved in the introduction or change of a desired property. There is no need to purify the modified material and thus there are no problems in losses during purification's. This again enables an easy verification of the secondary structure. Therefore this invention is an improvement as compared to molecular biological techniques because the approach is more convenient, better verifiable and faster than the molecular biological approach.

Modified growth factors:

Superaonists:

In many structure-function research efforts on the various growth factors, muteins have been found that have an improved activity. For instance in the European patent application EP-131816 the described goal is to obtain a beta-Interferon with an improved biological activity and/or a less side-reactivity. Also various examples of chemical modifications are available: for instance the European application EP 236987 describes the modification of IL-2 to obtain a less toxic and less immunogenic substance with improved kinetics in clearing from the body. Patent application EP-0442724 describes

PEG-ylated IL-6 which is a product with a longer half-life and an enhanced biological activity. Patent application WO 88/01511 describes succinylation of IL-2 whereby an enhanced solubility is achieved. In non of these patent applications there is any modification strategy besides a trial and error approach in which random mild modification is used to obtain one or a few modifications on the molecule. In addition, substantial losses in purification can occur, which was also the case in the example of

PEG-ylation of IL-6 where only a 10% yield of the desired protein was obtained. The localization of the modification was done in none of these inventions.

In present invention there is almost no loss and the localization of the modification can well be performed. In addition, gradual chemical modification makes it even possible to specifically modify 1 residue at I place in the molecule, as demonstrated in the examples I and 2-Although most modifications are performed with irreversible agents, also reversible agents can be used. This also enables the very specific modification of other residues.

Antagonists:

In the European patent application EP-0413383AI an antagonistic effect of a human IL-3 mutant is mentioned. However in this case it concerns the relation of remaining biological activity as compared to its receptor binding capacity. Thus, a real suppression the activitive of the native IL-3 was not shown.

In patent applications PCTIUS93/11197 and PCT/US93/11198 al kinds of IL-3 mutants are claimed, but also in this case there is no support what so ever of a real inhibitory activity. In addition, it is not probable that the mutants with the lowest activity are also the best inhibitors, since the chance on structural distortion is much higher than the chance on specific elimination of the catalytic activity.

There are other real antagonists for receptors of bovine growth hormone (Endocrinology 130: 2284 (1992)), mouse interleukin-2(EMBO J. 11: 3905 (1992)) Golan hepatocyte growth factor (Biochemistry 31: 9555(1995)), IL-I(Scand. J. Immuro!. 36: 27(1992)) and IL-4 (J. Exp. Med. 178: 2213 (1993). In all these publications no modification strategy was described; the antagonists were all by-products of structure-function analysis research. In addition, the concentrations of inhibitor that were needed to obtain significant inhibition were on average a hundred fold higher than the native growth factor concentration. These are concentrations that are harmful to clinical value of these antagonists.

The only strategy to generate a growth factor antagonist has been used for human growth hormone (Science 256: 1677(1992)). This was based on the disruption of one of the two receptor binding sites of the hormone. Also in this case there was a decrease of a factor 50 in receptor binding capacity, therefore requiring a problematic excess of inhibitor as compared to growth factor.

However, with present invention it is possible to obtain a clinically applicable inhibition, possibly even with an enhanced receptor binding capacity. This can be explained with the hypothesis that the growth factor itself performs some kind of catalytic activity after receptor binding. This hypothesis was supported by the fact that IL-3 contains a catalytic zinc-ion (Biochem. Biophys. Res. Commun. 187: 859 (1992)). It is not necessary that the growth factor contains complete catalytic center. It is very well possible that the catalytic center is completed only in the growth factor -receptor binding. Therefore the concerning chemical modification is directed as specific as possible towards residues that are directly or indirectly involved in such Zinc binding and/or catalysis and this without distortion of receptor binding ability. The starting material can be any protein or peptide containing substance. However, because the Zinc ions can protect against denaturation, it might be necessary to reversibly denaturate the molecule and to add chelating agents to remove this Zinc from the molecule. As an illustration, but not as an limitation of the invention the modification of IL-3 with lodo-acetate is described. At the concerning pH this modification is directed to alkylation of His-residues. The method however, can be performed easily also with other reagents by a person that is skilled in the art. The same can be stated about the modification of other amino acids that are involved in the catalytic activity and/or Zinc binding. Also these residues can be easily modified by other chemical ways or by molecular biological ways, for instance by using deletion or substitution mutations. In addition this is not limited to IL-3 alone: There is a significant homology among the various receptors of the cytokine-superfamily: For instance Interleukins 2-7 Epo and GM-CSF. On top of that specific Zinc binding has also been found for IL-2 IL-6 GM-CSF and gamma IFN. It is also conceivable that the invention can be applied to much more signal peptides and/or proteins, since for instance Insulin, human growth hormone and prolactin also have specific Zinc binding properties. In addition, it has been found that for certain cell-lines IL-3 can be replaced by for instance Insulin. Finafly it is possible that alkylation of a growth factor also has an other mechanism in the generation of a antagonist or cell-inhibitor. Therefore, in general also alkylation, preferably with Iodo-acetate, is to be regarded as a separate approach in the invention. Therefore all the above mentioned applications and/or modified substances and/or DNA-constructs and the use thereof are embodied within the scope of the invention.

Therapies against AIDS:

As already discussed, the invention can possibly be applied to the fight against AIDS. It should provide a better way to combat AIDS than the current possibilities:

In the patent USA 5215745 of Csatary et al. an aspecific method for immunotherapy against AIDS is described. In this case a means of an aspecific virus infection of avian paramyxovirus and/or avian rotavirus is used to enhance the number of CD4 positive cells. This can at best lead to a postponement of the disease because the newly formed CD4 positive cells will be infected by the HIV after a short time. In contrast, our invention leads to the effective cellular immune-response against retroviruses. In patent number USA. 5081226 of Berzofsky et al. a therapy is directed at a specific immune-response against retroviruses. In this case for instance antibodies against the HIV glyroprotein 160/120 complex are generated. In our research that corresponded to the invention this approach lead to the protection of the HIV and its integration in histiocytic cells, thereby even promoting the disease. As a result the method could not demonstrate efficacy.

For this reason the patent number USA 5256767 describes a virus subunit vaccine without envelope. The draw back of this method however, is the fact that lipophylic nuclear parts can at best express in a very low concentration in the context of MHC and therefore do not provide sufficient protection. Vaccines that are based on inactivated viruses combine the objections of the two aforementioned patents.

In contrast to the above described problems we describe in our example 7 that HTLV_{IIIB} (AIDS Research and Reference Reagent Program Catalog, NIH Publication No. 91-1536, Bethesda, MD, USA) is eliminated in vivo at low levels of antibodies. From the oligocibnal swarm HTLVIIIB, it is known that it causes a persisting retroviral infection in normal circumstances. This also was demonstrated by the HTLVIIIB infection of coworkers within the group of John Moore via a needle accident. (AIDS Res. Hum.

Retrovir. 6: 307 (1990) and J. Clin. Invest. 91: 1987 (1992)). In contrast from our example it can be deduced that these viruses can be eliminated if the antibody levels are low. The generation of B-cells and the subsequent antibody production by these B-cells is stimulated by various growth factors (for instance IL-1-7 and IL-I 1-). Therefore it is logical that the therapy against AIDS can also be accomplished by using antagonists of these growth factors. Thus, also the treatment of ADS is considered to be one of the applications of the invention.

### EXAMPLES

### Example 1

Gradual chemical modification of IL-3 with Acetic Anhydride to obtain an IL-3 with an enhanced biological activity or a changed stability.

Materials and Methods:

Chemical Modification:

Acetic Acid, dioxane, lysine hydrochloride and MES were from Sigma. the modifying agent was from Fluka.

Buffers: Acetate/NaOH was used for modification at pH 5.0, MES/NaOH was used for modification at pH 5.5, 6.0 and 6.5. At pH 7.0 an NaH₂PO₄ / NaOH buffer was used. Ten times concentrated stock solutions were prepared and directly filtered trough a 0.22 micron filter.

The reaction mixture contained 50 mM buffer, 2 mglrnl hIL-3 and 3 mnM Acetic anhydride or Succinic anhydride respectively. The 10 times concentrated stock solutions were prepared fresh at the day of the experiment. Modification of hIL-3 was performed overnight at 30 ° C. After modification it was determined by SDS-electrophoresis that the IL-3 was not degraded.

Tests of biological activity:

MO-7 cells were a kind gift of dr. I.P. Touw (Erasmus University of Rotterdam, The Netherlands). RPMI culture medium was from Gibco (Paisly, UK.). supplemented Bovine Calf Serum was from Hyclone (Logan, Utah, USA.) The cell culture medium consisted of RPMI with 10% of the calf serum. During normal tissue culture of the cells also 100 ng/ml IL-3 was added.

Firstly 10 fold stock solutions were prepared consisting of serial 3 fold dilution's ranging from 10 microgram/ml to I ng/ml in cell culture medium. after mixing thoroughly, 25 microliter of the stock solution was added to 225 microliter cell culture medium, followed by incubation at 37 ° C. For the 6-day tissue culture 2 x 10 M07 cells ml were used as an end concentration and for the 10 day culture 4+10³ MO7 cells/ml were used. After the tissue culture, overnight tritium thymidine incorporation was used to determine biological activities. From at least 2 independent growth-response curves there was a determination of the average (and range of ) the concentrations that resulted in 50 % maximal stimulation. The relative activity of the modified substances was determined as the ratio of these 50% concentrations of the modified and the native IL-3 respectively ([IL-³modified]⁵⁰% 1 [IL-³native]⁵⁰% ).

The standard activity for the native IL-3 on day 6 was 1.0 million units/mg of protein (n=l0, sigma(n-1)=20%). On day 10 it was 0.2 million units/mg of protein (n=10, , sigrna(n−1)=30%).

Results:

All results regarding to the more precise characterization in terms of average number of groups, the specificity and the places of the modification on the molecule are described later in this patent description. The results of the biological tests are shown in table 1:

TABLE I
Relative biological activities of acetylated IL-3.

Average relative activity
(and activity-range) in tissue culture

| Acetylation at | After 6 days | | After 10 days | |
|---|---|---|---|---|
| pH = 5.0 | 1.5 | (1.4-2.1) | 1.4 | (0.8-1.5) |
| pH = 6.0 | 0.9 | (0.8-1.0) | 0.9 | (0.7-1.1) |
| pH = 6.5 | 0.5 | (0.4-0.6) | 0.2 | (0.2-0.3) |
| pH = 7.0 | 0.9 | (0.9—0.9) | 0.6 | (0.6—0.6) |

From the table it can be concluded that there is a significant difference in relative activity between day 6 and day 10 after modification at pH 6.5 and pH 7.0. Therefore, it can be assumed that this demonstrates the generation of a substance with significantly lowered stability. At pH 5 there might be an enhancement of biologic activity. Further aspects are discussed in examples 5 and 6.

### Example 2

Gradual chemical modification of IL-3 with Succinic anhydride for the generation of an improved IL-3 with an enhanced activity or stability.

Chernical Modification

Acetic Acid, dioxane, lysine hydrochloride and MES were from Sigma. the modifying agent was from Fluka. Buffers: Acetate/NaOH was used for modification at pH 5.0, MES/NaOH was used for modification at pH 5.5, 6.0 and 6.5. At pH 7.0 an NaH₂PO4 / NaOH buffer was used. Ten times concentrated stock solutions were prepared and directly filtered trough a 0.22 micron filter.

The reaction mixture contained 50 mM buffer, 2 mg/ml hIL-3 and 3 mM Acetic anhydride or Succinic anhydride respectively. The 10 times concentrated stock solutions were prepared fresh at the day of the experiment. Modification of hIL-3 was performed overnight at 30 OC. After modification it was determined by SDS-electrophoresis that the IL-3 was not degraded.

Results

All results regarding to the more precise characterization in terms of average number of groups, the specificity and the places of the modification on the molecule are described later in this patent description. The results of the biological tests ( as performed in example 4) are shown in table 2:

TABLE 2
Relative biological activities of Succinylated IL-3.

Average relative activity
(and activity-range) in tissue culture

| Succinylation at | After 6 days | | After 10 days | |
|---|---|---|---|---|
| pH = 5.0 | 1.7 | (1.6-2.0) | 1.6 | (1.3-2.5) |
| pH = 6.0 | 1.4 | (1.2-1.8) | 1.3 | (1.3-1.4) |
| pH = 6.5 | 0.4 | (0.4-0.5) | 0.4 | (0.4-0.5) |
| pH = 7.0 | 0.3 | (0.3—0.3) | 0.3 | (0.2-0.3) |

From table 2 it can be concluded that succinylation at pH 5 results in a significant enhancement of the activity and succinylation at a pH=>6 results in a lower activity.

### Example 3

A method to chemically modify biologically active peptides or proteins for the generation of a protein or peptide antagonist.

Materials and methods:

Urea, EDTA, MES and NaOH were from Sigma. the Na-lodo-acetate was from Fluka. Buffers: MES/NaOH was used for modification at pH 6.0. Ten times concentrated stock solutions were prepared in 8 M urea and filter-sterilized directly thereafter with a 0.22 micron filter. The reaction mixture contained 50 mM buffer, 5.5 M urea and 50 mM EDTA. From these reagents 10 times concentrated stock solutions in 8 M urea were made freshly on the day of the experiment.

1. Moderate chemical modification of hIL-3: lodo-acetate was added in a concentration of 3, 10 and 30 mM. The IL-3 concentration was 2 mg/ml. The modification was performed during 24,48 and 72 hours at 37 OC and subsequently it was studied by native electrophoresis. After 2 days and 30 mM lodo- acetate there was maximal modification without severe distortion of the bands from the modified material (an indication of severe denaturation of the molecules). In this case less than 2% of the starting material was left. Because in this case there was an expectation of a minimal biological activity without severe denaturation of the protein, this sample was used in further experiments. Subsequently, SDS electrophoresis was used to demonstrate that no degradation of the molecule was found after modification.

2. Partial chemical modification: In order to optimize the inhibitory capacity of the modified hIL-3, also partial modification was performed. For this purpose I mg/ml IL-3 was modified for 18 hours at 37° C. with 10, 30 and 100 mM of lodo-acetate. After native electrophoresis and coomassie staining the sample of 100 mM gave maximal modification without excessive distortion of the bands in the gel. circa 5% of the starting material was still present.

Since only this sample showed inhibitory activity in the activity tests, this sample was used in further experiments.

3. The activity- and inhibition assays:

The activity tests were performed as described in example 1. Growth response curves (n>=2) for both controls and alkylated IL-3 were made by 10 fold serial dilution's ranging from 1000 to I nglml. Inhibitory activity of the alkylated IL-3 was tested by performing the same titration of control L-3, but now in presence of 3 ng/ml alkylated IL-3.

In order to determine the maximal receptor binding capacity partially modified IL-3 was titrated in serial 7 fold dilution in presence of 3 ng/ml of native IL-3. The titration range was from 15gg/ml to 0.1 ng/ml and the titration was performed on only 4000 M07 cells/ml to exclude an starvation phenomena's.

Results:

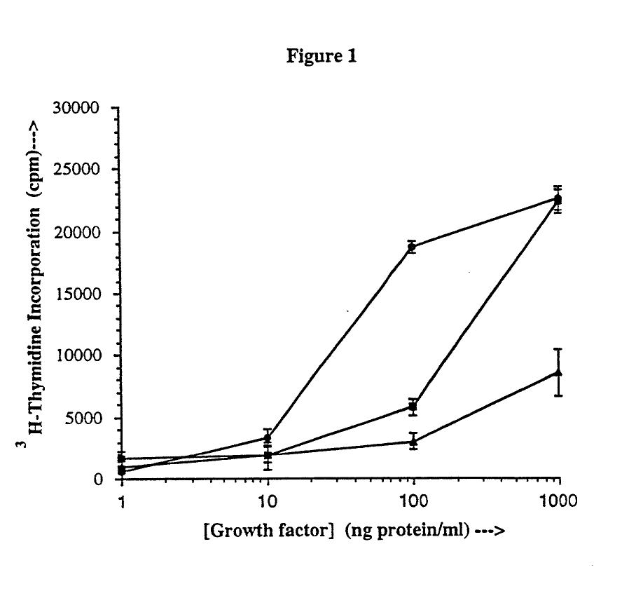

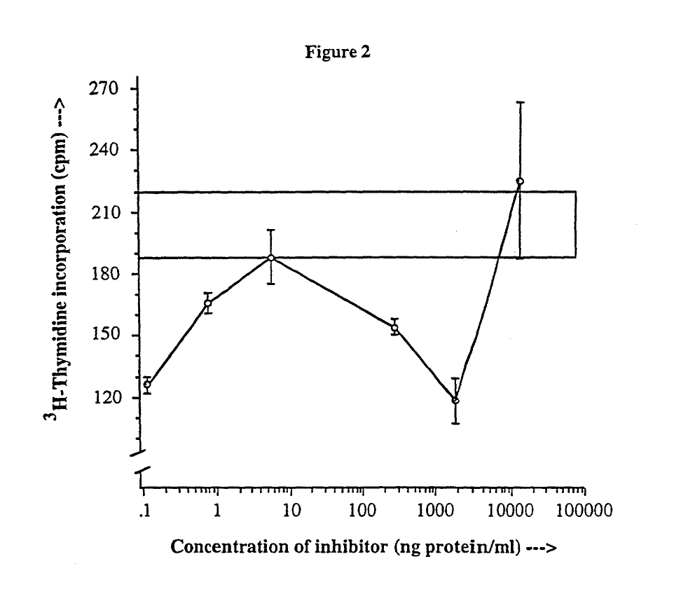

In FIG. 1 it is shown that the modified IL-3 is capable to inhibit the control-IL-3 by a factor 10-100. In addition 3 ng/ml of the modified IL-3 is able to suppress thymidine incorporation of 30-100 ng/ml control IL-3 for 80-90%. Therefore, the modified IL-3 does not only have an Inhibitory activity, it also has an enhanced receptor binding capacity. This is confirmed in the titration of partially modified IL-3 (FIG. 2): Only 0.1 ng of partially modified IL-3 is sufficient for almost 50% inhibition of 3 ng/ ml native IL-3.

### Example 4

Method for gradual enzymatic exoprotease treatment of IL-3 for the generation of a modified IEL-3 with a changed stability and/or activity.

Materials and methods.

Exoprotease treatments have been performed with Cathepsine-C and with Carboxypeptidase-Y from Boehringer. I mg/ml of EL-3 was incubated for 18 hours at 37° C. in presence of the protease. Cathepsine-C was added in serial two fold dilution's in a range of ½to 1/128mg/ml. The other reaction conditions were as described by the manufacturer.

Biological activities have been determined as described in example 1.

Results

The approach lead to the results in table 3. The modifications that did not result in a change in biological activity are not shown:

TABLE 3
Changed activities after gradual exoprotease treatment:

| | | Average relative activity | | | |
|---|---|---|---|---|---|
| Protease | [Protease] | (and range) in tissue culture | | | |
| treatment | (mg/ml) | After 6 days | | After 10 days | |
| Carboxy- | 1/40 | 0.6 | (0.5-0.7) | 1.0 | (0.7-1.5) |
| peptidase Y | 1/20 | 0.08 | (0.07-0.10) | 0.2 | (0.13-0.21) |
| | 1/10 | <0.05 | (<0.05) | <0.05 | (<0.05) |
| Cathepsine C | 1/32 | 2 | (2-6) | 1.0 | (0.7-1.5) |
| | 1/16 | 5 | (3-6) | 1.0 | (0.9-1.1) |
| | 1/8 | 3 | (2-4) | 1.3 | (0.9-1.7) |
| | 1/4 | 3 | (2-4) | 1.3 | (0.9-2.1) |

From this table it can be concluded that treatment with Carboxypeptidase Y at a concentration of 1/40and at 1/20results in a substance with a relative activity that is lower at day 6 than at day 10. Therefore, this indicates a substance with a higher stability as compared to native IL-3.

It can also be concluded form the table that Cathepsine C significantly enhances the activity at day 6 but not at day 10. Therefore, this is a also substance with a lowered stability.

### Example 5

Localization of modifications in a peptide or protein by the combination of protease treatment and mass spectrometry.

Materials and methods:

Protease treatment:

For localization of the modified residues the modified material was dialyzed against the appropriate buffer and subsequently fragmented by Endo Glu-C or Endo Lys-C as described by the manufacturer (Boehringer Mannheim, Germany). The incubation was overnight at 37 ° C., 2 mg/ml hIL-3 and a protein to protease ratio of 30.

Laser Desorption Mass Spectrometry (LDMS):

Pre treatment

Solutions of 2,5 dihydroxybenzoic acid (DHB, Mr=154.12; 10 gil) in milli Q water were made freshly before each experiment. Both the (un)modified IL-3 solutions and their digests were diluted to 0.1 mg/ml. From these diluted solutions 0.5 μI was mixed with 0.5 μl DHB-solution on the target. Subsequently, the target was dried to air at room temperature in a slow stream of air.

Mass Spectrometry:

Matrix Assisted Laser Desorption mass spectrometry was performed on a Finnegan MAT Vision 200 laser desorption mass spectrometer, equipped with a pulsed nitrogen laser (337 nm, pulse width 3 ns). The sample was exited to just above the ionization threshold (10⁶-10⁷ W/cm²). The acceleration voltage was 6.5 kV. The ions were post accelerated to the conversion dynode on - 10 kV for the electron amplification. Standard accuracy was about 0.05%, but this can deteriorate to 0.1-0.2% due to experimental conditions.

Results

Because the signal of the LDMS was still sufficient in spite of the higher molecular weights, it became possible to localize all modifications. Two examples are shown:

1-Localization of modification with Succinic anhydride (pH 5.0) by means of Endo Lys-C treatment and LDMS: At the pH=5.0, Succinic anhydride modification and subsequent Endo Lys-C digestion a peak shifted from 1085 d to 1 185 d, also the peak at 1108 d (1085+23 from Na⁺) shifted to 1208 d. Based on the protease specificity and the amino acid sequence this 1085 d peak can only correspond Ala¹- Lys¹⁰. Since modification of LysIO would disable the digestion on this amino acid, there would not be any Ala¹- Lys¹⁰-fragrnent at all. Therefore, the modified amine residue is the amino terminus Ala¹.

2-Detection of modification of Lvs²⁸ A with Acetic anhydride by both Endo Glu—C treatment with subsequent LDMS and Endo Lys—C treatment with subsequent LDMS:

At the pH7 Acetic anhydride modification and subsequent Endo Glu-C treatment the peak at 1598 d shifted towards 1640 d. This shift corresponded exactly with the mass of 1 acetyl group. Also in this case the amino acid sequence enabled the localization to Ile²³ - Asp³⁶ . Since Lys 28 is the only amine-residue in this fragment, it can be deduced that this is the modified residue. This was confirmed by the Endo-Lys digestion where a fragment of 5815 d emerged. This fragment can only be explained if Lys²⁸ is the modified residue, thus disabling the digestion after that residue.

The other modifications have been analyzed in a similar way, resulting in table 4:

TABLE 4
Localization of modifications on IL-3:

| | Number of modified groups after modification with | | | |
|---|---|---|---|---|
| Modified at pH: | Acetic anhydride | | Succinic anhydride | |
| 5.0 | Ala¹ | >90% | Ala¹ | >90% |
| 6.0 | Ala¹ | >90% | Ala¹ | >90% |
| | Lys²⁸ | ±45% | Lys²⁸ | >45% |
| | Lys⁶⁶ | ±20% | Lys⁶⁶ | ±20% |
| | Lys¹⁰⁰ | ±40% | Lys¹⁰⁰ | ±25% |
| | Lys¹¹⁶ | ±40% | Lys¹¹⁶ | ±40% |
| 6.5 | Ala¹ | >90% | Ala¹ | >90% |
| | Lys²⁸ | ±70% | Lys²⁸ | ±55% |
| | Lys⁶⁶ | ±50% | Lys⁶⁶ | ±40% |
| | Lys¹⁰⁰ | ±65% | Lys¹⁰⁰ | ±50% |
| | Lys¹¹⁶ | ±80% | Lys¹¹⁶ | ±90% |
| 7.0 | Ala¹ | >90% | Ala¹ | >90% |
| | Lys¹⁰ | ±20% | | |
| | Lys²⁸ | ±55% | Lys²⁸ | ±70% |
| | Lys⁶⁶ | ±35% | Lys⁶⁶ | ±40% |
| | Lys¹⁰⁰ | ±65% | Lys¹⁰⁰ | ±70% |
| | Lys¹¹⁶ | ±40% | Lys¹¹⁶ | ±90% |

The table shows that both modifications have the same target residues on the protein. The only exceptions are that Acetic anhydride has a slightly higher degree of modification at pH>=5.5 and at pH 7 Lys¹⁰ was partly modified in contrast to the Lys¹⁰ of the Succinic anhydride modified material.

Table 4 also shows that Lys¹¹⁶ is at least partly protected at pH=7. Since a phosphate buffer was used at that pH, the possibility arises that a phosphate group is binding at that place and thereby shields the Lys¹¹⁶ for modification. To test this hypothesis hIL-3 was modified at 50 mM buffering substance, consisting of MES and Phosphate. Acetic anhydride (1, 2 and 3 mM respectively) was used for the modification at pH 6.8 which is well within the buffering range of both buffers. In presence of 10 mM or more phosphate there was protection of 1 group: Lys^{116.} At a phosphate concentration below I mM this protection was absent. Since 10 mM is the physiological phosphate concentration, it can be assumed that present localization method enabled the demonstration and localization of a biologically significant phosphate binding. Therefore it is very conceivable that an antagonist or cell-growth inhibitor can be generated by distortion of this phosphate binding. Therefore, also this is to be considered within the scope of the invention. It can also be stated that the residues Lys²⁸ and Lys⁶⁶ also had slight protection by the phosphate, suggesting a close proximity in the 3-D structure. Thus, in this way it can even provide structural information.

Finally it can also be stated that the gradual chemical modification can be performed with such minimal degree that a specificity can be accomplished that is not limited to some types of-residues, not to only amine-residues, but even to I amine-residue on the complete molecule, namely Alai. Therefore this specificity is also included in the claims.

### Example 6

Quantitative structure function analysis research using gradual chemical modification, protease treatment and laser desorption mass spectrometry.

Materials and methods:

QSAR-strategy:

This example was demonstrated with lysine modifications on hIL-3. The strategy consists of 5 steps of which the first step concerns the gradual chemical modification of the protein. Although the micro-environment of the various residues in the 3-D structure is not known, differences can be expected on the amino acid sequence alone. Even more differences can b expected in the 3-D structure.

We investigated acylation reactions on hIL-3 (step 1). These reactions onlv take place on uncharged Lys residues, enabling the gradual modification by a stepwise increase in the pH of the modification-reactions.

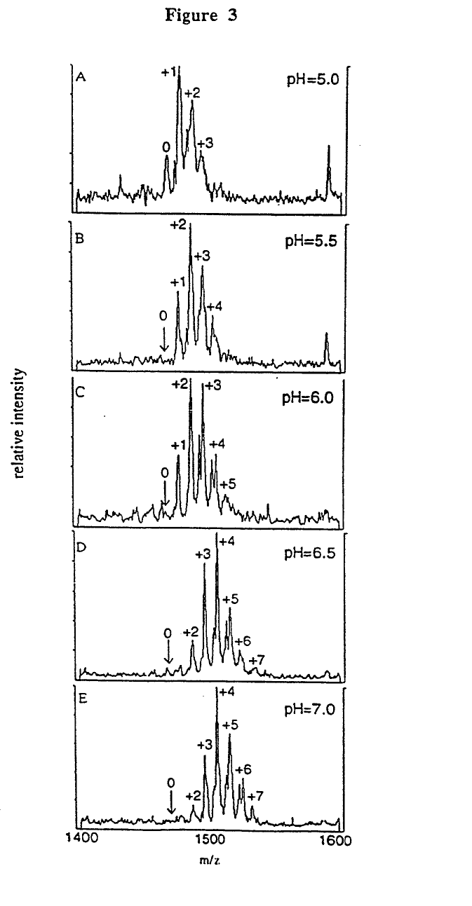

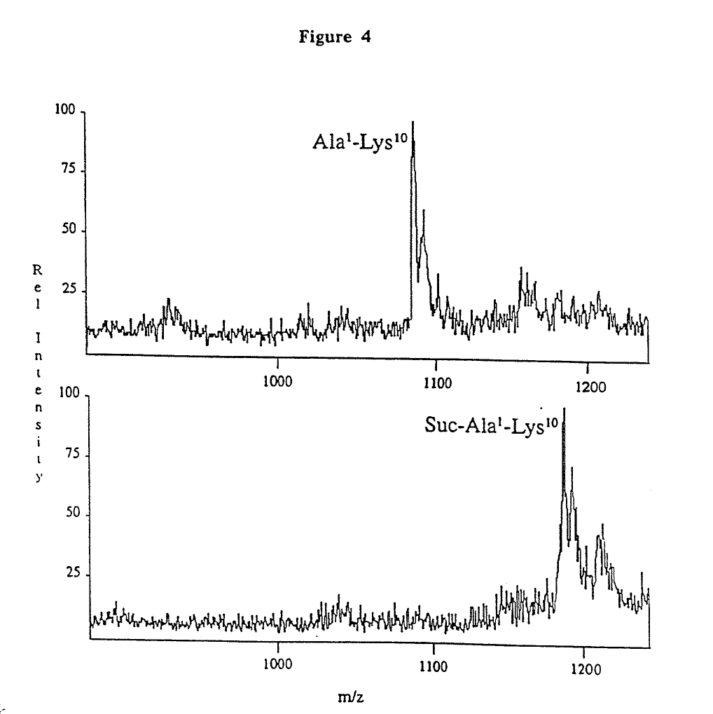

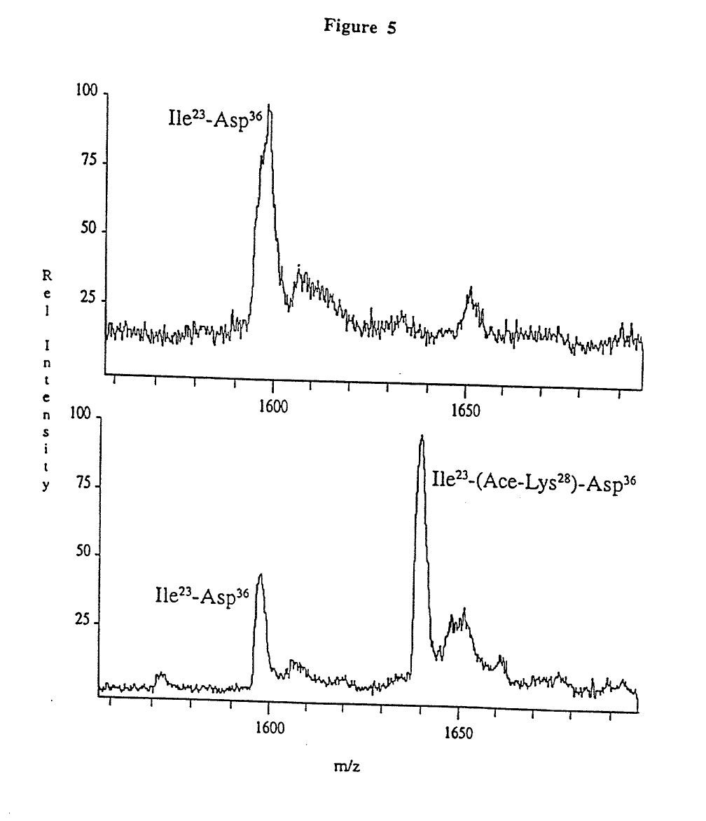

The second step is the monitoring of the modification reaction. In order to study a sufficient number of possible conditions so that the optimal conditions can be achieved, a mild and sensitive method is needed. This method is native electrophoresis (Electrophoresis 15: 251 (1994)). However, also electrospray mass spectrometry can form a suitable alternative. This is demonstrated in FIG. 3: Electrospray Mass Spectrometry of Succinylated EL-3 at pH 5-7. Especially the combination of both enables the demonstration of complete specificity on amine-residues.

The third step is the confirmation of the overall structural integrity. Circular Dichroism Spectroscopy can be used for this purpose(Electrophoresis 15: 251 (1994)). Although small differences are not visible by this method, substantial structural changes like denaturation are clearly detected.

The fourth step is the characterization and localization of the modified residues, for which the following techniques were used: native digestion with specific protease's, electrophoresis, electrospray mass spectrometry, and LDMS. The reaction specificity was determined by the combination of native electrophoresis and electrospray mass spectrometry. Localization was performed with endoprotease's and LDMS, as described in the previous example.

The fifth and last step is the testing of biological activity of the various modified forms of the protein. After this determination of activity the real involvement of the various localized residues can be deduced.

Results

Chemical modification, structural confirmation and monitoring of the reaction: Chemical modification of hIL-3 and the monitoring was performed with Succinic anhydride or Acetic anhydride as previously described. Subsequently, it was found by Circular Dichroism that Succinic anhydride modifications at a pH larger than 7 resulted in an overall structural change (denaturation). Therefore, only the modifications at or below pH 7 were used for further investigation.

Characterization and localization of the modifications:

See previous example.

Activity tests of the various modified forms of the protein and localization of biologically important residues:

Both the methods and the results of the tests for biological activity are described in examples 1 and 2. The combination of these results and the results of the localization of the modified residues ( Tables I and 2 and previous example) enables statements on involvement of several residues. Hereby, an important change is from unmodified to modified at pH 5, which is accompanied by an enhancement in activity. Other important changes are from pH 6 to pH 6.5 (activity-decrease by a factor 2) and from 6.5 to 7 (activity increase by a factor 2):

Since Succinic anhydride modification at pH 5 is accompanied by the modification of only 1 group, namely the amino terminus (Ala¹), it can be concluded that this group has some kind of limiting or regulating action. The increase in activity has also been found in structure-function research with deletion mutants.(J. Biol. Chem. 266, 21310 (1991); Proc. Natl. Acad. Sci. USA. 89: 11842(1992)), but it was never assigned to the first residue alone.

With the differences between pH 6 and pH 6.5 there is a more complex pattern: For acetic anhydride the decrease in activity of a factor 24 was accompanied with a modification of Lys²⁸ of 45% to 70% of the groups, for Lys⁶⁶ of 20% tO 50% of the groups and for Lysioo from 40% to 65% of the groups. Finally, there occurred modification on Lys, ¹⁶ of 40% to 80% of the groups, which is a decrease of unmodified groups of 60% to 20% : a factor 3 difference. Since this difference correlates exactly with the activity decrease, Lys¹¹⁶ is the best candidate for the biological activity. This is confirmed by the factor 3 lowered modification at pH 7 (in comparison with pH 6.5), that is accompanied with a factor 3 increase in activity. Therefore, Lys¹¹⁶ is important for biological activity. This was all confirmed by modification with Succinic anhydride. In this case there was no decrease in modification for the at pH 7 modified material as compared to the at pH 6.5 modified material. Accompanying this phenomenon there was no enhancement of activity either.

Thus it has been demonstrated that the residues Lys¹¹⁶ and the amino-terminus are of biological significance, while the amino terminus seems to have an inhibitory or regulating influence, Lys¹¹⁶ seems to be important for the biological activity. In addition, Lys¹¹⁶ is also protected by phosphate, suggesting a phosphate binding by that residue. Since the residue is also important for the biological activity of the interleukin, this suggests that phosphate-binding is of importance for the mode of action of IL-3 and if this process is of importance for IL-3, it can also be of importance for other peptides and proteins.

Summarized, this method enables the localization of biologically important residues and the demonstrated phosphate binding has also enabled the establishment and localization of a possibly important physiological process. Therefore, the invention also embodies the modification of a protein or peptide to introduce a new, preferably antagonistic activity by means of the manipulation of the phosphate binding of the protein or peptide.

### Example 7

Lowered levels of antibodies lead to effective in vivo cellular resistance.

Materials and Methods:

The test system consisted of human chimeric 4 week old “X-linked immunodeficient” Mice . the chimerism was induced with conditioning by total body irradiation (TBI), and transplantation of 4 million human peripheral blood lymphocytes/gram of recipient.

The TBI of the CBA/N mice was 9 Gy gamma. These mice also received a blood supporting treatment in the form of 0.5 million autologous bone-marrow cells intravenously (iv). Comparable irradiation, processing of the human blood and the transplantation is described in the Eur. J. Immunol. 22: 197 (1992). The mice were injected intraperitoneally (ip) daily with 10.000 I.U. of human Interleukin-2 (Eurocetus, Amstelveen, Benelux). The infection was done ip I hour after the transplantation of the human cells with a dose that is 10 times the minimal dose, still infectious in the “infectious center test” or ICT.

The conditioned CBA/N mice were pretreated ip with 250 microgram of monoclonal antibody anti- HIV-1GP13 (against the CD4-binding place) or the anti HTLV_{IIIB} F58H3 directed at the V₃-loop.

The ICT was performed in duplo with CB 15 cells (Proc. Natl. Acad. Sci. USA. 89:3116 (1992)). Ten thousand cells were plated per well and after 5-7 days an ELISA was performed on HIV p24 protein (Organon Technica, Oss, The Netherlands). The sacrifice of the animal was always within 2 weeks after transplantation thus eloping the production of antibodies (unpublished data). On the day of the sacrifice of the mice, the cells were rinsed from the peritoneal cavity with medium that contained heparin (Organon Technica, Oss, The Netherlands). On these cells the ICT was performed in presence of CB15 cells and 100 I.U. human Interleukin-2 /ml of culture medium.

Titration was performed in duplo in a titration range from 2.5 million to 0 in serial 5 fold dilution's. After 5-7 days the ELISA-test was performed on the HIV-p24 protein in the supernatant of the culture medium . As a control for the presence of CD4⁺cells FACScan analysis was performed as described in the Eur. J. Immunol. 22: 197 (1992).

Results

The results are shown in Table 5:

TABLE 5
Protection of HIV_{IIIB} virus by antibodies:

| | 1/IC × 10⁴ | | | |
|---|---|---|---|---|
| | at day 5 after transplantation | | at day 8 after transplantation | |
| Antibody | Average | (Range) | Average | (Range) |
| none | 2 | (0.4-) | >200 | >200 |
| GP13 | 4 | (0.4-100) | 20 | (2-50) |
| F58H3 | >200 | (>200) | =>200 | (0.5->200) |

The table shows that HTLV_{IIIB} persists for the first 5 days under these circumstances. However 8 days after the transplantation it appears to have been eliminated, even in abundant presence of human CD4+cells. This indicates that the transplanted human T- cells eliminate the virus. However if either of the specific anti-HIV-1 antibodies are administered to the chimeric mice, the virus did persist (Table 5), demonstrating that the persistence of HIV-infection is caused by antibodies. From this it can be deduced that the lowering of the antibody levels in HIV infected humans can enable the T-cells to eliminate the virus, thereby providing the cure of the infection. These antibody-levels can be lowered by suppression of B-cells. Therefore this B-cell suppression is an interesting field of applications for the growth factor antagonists. There are also other possibilities for the suppression of the HIV infection which can also be used separately:

1. Plasmaphoresis, that results in the lowering of the antibody-level. The usual clinical practice is the complete substitution of the plasma. An experimental therapy for instance for myasthenia gravis is the so called selective recovery of the plasma. In this case the plasma of the patient is purified from the harmful antibodies before returning to the patient. This in vitro selection can also be used for HIV-reactive, by preference HIV-envelope reactive antibodies.

2. Leukophoresis, f6r the lowering of the number of B-cells. It is preferred to remove the B-cells that are HIV-reactive. The leukocytes can be removed totally from the HIV-infected person. This form of leukophoresis is a routine clinical practice for other diseases. For HIV infected persons however. it has never been described. Alternatively the selective return of the white blood cells without B- cells can very easily be done. Selection can also consist of positive selection for T-cells or sub populations thereof.

3. In vivo depletion of antibodies. The invention also includes in vivo depletion by formation of immune complexes, as well selective as non-selective. Non selective removal is preferably done by antibody-specific antibodies. Selective removal is preferably done by virus, inactivated virus, virus-subunits and/or virus-like or identical proteins or peptides. These substances are preferably coupled to substances that promote clearing from the body.

4. In vivo depletion of B-cells. This in vivo depletion can be performed trough non-selective removal with B-cell specific antibodies, by preference with B-cell apoptose-inducing antibodies. This can also be performed with bi-specific antibodies, preferably of the combination CD 19 /CD3-reactivity. This has already been used in a phase I clinical trial in the Academic Hospital Utrecht (Utrecht, The Netherlands) for patients with B-cell tumors. This therapy results in a very substantial depletion of the number of B-cells (Personal communication: F.A. van Houten Academic Hospital Utrecht, The Netherlands). Selective removal of B- cells is preferably done by virus, inactivated virus, virus-subunits and/or virus- like or identical proteins or peptides or by antibodies. Preferably, these substances are coupled to B-cell depletion promoting substances.

5. Other methods that suppress the in vivo production of antibodies. An example is the use of transforming growth factor beta (TGF-beta).

HIV and other viruses integrate as provirus in the host genome. Therefore it can be present in these cells in al latent state for a long time. Therefore, in this invention it is preferred to activate such a provirus, preferably by administration of IL-2 to the host.

HIV persists in the histiocytic cells and these can produce low concentrations of virus that might escape recognition by the hosts immune-system. Therefore it is preferred to prolong the treatment for at least the life-time of these cells. In addition it is preferable to simultaneously perform passive immune-therapy, preferably with immunoglobulins of subjects that are not HIV-infected. Therefore, this passive immune therapy is to be considered within the scope of the invention.

Although this invention is described in a manner that is based on the limited knowledge of retroviruses like HIV, it is clear that several modifications can be made without diverting from the scope of the invention.

What is claimed is:

1. A method for chemical modification of human Interleukin-3, preferably for the introduction of one or more of the following features: enhanced biological activity, enhanced stability, suppressed antigenecity, acquired antagonistic activity or cell inhibitory activity.

2. A method according to claim 1, wherein the modification is a gradual modification, preferably under gradual varying conditions, wherein one or more of the following conditions are varied: pH between 5.0 and 7.0, preferably in steps of 0.5 pH units, and/or time or reagent-concentrations.

3. A method according to one or more of the preceding claims, wherein the substrate is not human IL-3 but one or more of the following preferably human proteins or peptides: Other Interleukins, heamopoietic growth factors, peptide hormones or protein hormones, signal peptides or signal proteins, biologically active proteins or peptides.

4. A method according to one or more of the preceding claims, wherein the antigenecity is lowered by shielding possible interactions of antigenic response inducing amino acids in the protein or peptide.

5. A method according to one or more of the preceding claims, wherein the stability is changed, preferably because of shielding possible interactions of amino acids that form a binding place for protease's.

6. A method according to one or more of the preceding claims, wherein the receptor binding of the peptide or protein is enhanced by shielding the residues that reduce this receptor binding.

7. A method according to one or more of the preceding claims, wherein the receptor binding of the peptide or protein is enhanced by the introduction of a new chemical interaction, preferably a charge, preferably a negative charge.

8. A method according to one or more of the preceding claims, wherein the modification is specific for a few types of amino acid, one type of amino acid, for instance amine-residues and/or even 1 amine-residue in the peptide or protein, for instance the N-terminus.

9. A method according to one or more of the preceding claims, wherein the modification has specificity to one or more residues that are involved in catalytic activity, preferably His-residues.

10. A method according to one or more of the preceding claims, wherein the modification has specificity to one or more residues that are involved in catalytic activity, preferably His-residues for the introduction of an antagonistic and/or cell inhibitory activity.

11. A method for the chemical or non chemical modification of proteins and peptides for the introduction of an antagonistic and/or cell inhibitory activity by disruption of phosphate binding.

12. A method for specific chemical modification of selected-amino acids on a peptide or protein using gradual chemical modification and reversible reagents.

13. A method for localizing chemically modified amino acids by native electrophoresis to determine change in charge, protease treatment and mass spectrometry, preferably laser desorption mass spectrometry.

14. A method for localizing biologically important residues on a protein or peptide, by chemical modification, preferably in a gradual manner, native electrophoresis, activity tests and localization of modified residues as described in previous claims.

15. A method for gradual chemical modification of biologically active proteins or peptides as described in one or more of the preceding claims, wherein the modification can be performed in a very specific manner by using previously described methods for localizing residues on a protein or peptide that are involved in biological activity.

16. Human Interleukin-3, modified only at one or more a. the following residues: Ala¹, His²⁶, Lys²⁸, Lys⁶⁶, His95, His⁹⁸, Lys¹⁰⁰, or Lys¹¹⁶.

17. Any preparation, containing a modified peptide or protein (both in mixed form and in chemically bound form) that is prepared according to one or more of the preceding claims.

18. A modified signal substance, preferably a protein hormone, peptide hormone, a growth factor, a heamopoietic growth factor, an Interferon, an interleukin and/or a colony stimulating factor wherein the modification is within or in close proximity to a partial or complete catalytic center.

19. A substance, as described in one or more of the preceding claims, wherein the catalytic activity is changed.

20. A substance, as described in one or more of the preceding claims, wherein the modification is withere is or in close proximity to a metal binding center, preferably a Zinc binding center.

21. A substance, as described in one or more of the preceding claims, wherein the metal ion is within or in close proximity to a catalytic center.

22. A substance, as described in one or more of the preceding claims, wherein the metal ion has a catalytic function in the unmodified substance.

23. A substance, as described in one or more of the preceding ciaims, wherein the metal binding properties have been changed.

24. A substance, as described in one or more of the preceding claims. wherein the affinity of the signal substance for the receptor has not decreased for more than a factor 10, has remained the same or has even been increased.

25. A substance, as described in one or more of the preceding claims, wherein an enhanced biological activity, antagonistic activity and/or cell inhibitory activity has been obtained.

26. A substance, as described in one or more of the preceding claims, wherein the modification is a modification of an amino acid. This can be a chemical modification, preferably an alkylation and or an acylation or molecular biological modification like a deletion mutation and/or a substitution mutation.

27. A substance, as described in one or more of the preceding claims, wherein the modified amino acid is involved in the binding of a metal ion, preferably a Histidine residue.

28. A substance, as described in one or more of the preceding claims, wherein the signal peptide is a Zinc binding signal peptide, preferably one or more of the following: IL-2, IL-3, IL-6, IFN-gamma, Growth Hormone, Prolactin and/or Insulin.

29. A substance, as described in one or more of the preceding claims, wherein the signal peptide is a growth factor with receptors from the same (cytokine) superfamily as the IL-3 receptor, preferably IL-2, IL-3, IL4, IL-5, IL-6, IL-7, GM-CSF and/or Epo.

30. A substance, as described in one or more of the preceding claims, wherein the substance has acquired a change in stability, preferably an enhanced stability.

31. A substance, as described in one or more of the preceding claims, wherein the substance has acquired a lowered stability, preferably in combination with an antagonistic activity.

32. DNA-constructs that contain the genetic code for the proteins and/or peptides as described in I or more of the preceding claims.

33. Any preparation containing one or more substances, (both in mixed form and in chemically bound form), that is described in one or more of the preceding claims or is prepared according to one or more of the preceding claims.

34. The use of any preparation as described in one or more of the preceding claims.

35. The use of any preparation as described in one or more of the preceding claims, preferably for one or more applications as described in the field of applications in this patent-description.

36. Inhibition, suppression and/or the cure of a HIV infection by suppression of antibody production by B-cells and/or the suppression of generation and/or maturation of B-cells, preferably by a preparation as described by one or more of the preceding claims.

37. A method and/or a product as described in, or in combination with, one or more of the oreding claims, wherein the antibody levels are lowered, preferably by piasmaphoresis, partial or complete plasma recovery or selective return of the serum.

38. A method and/or a product as described in, or in combination with, one or more of the preceding claims, wherein the selection is performed in vitro, preferably by removal of antibodies, preferably HIV-reactive antibodies, preferably HIV-envelope reactive antibodies.

39. A method and/or a product as described in, or in combination with, one or more of the preceding claims, wherein leukophoresis is performed.

40. A method and/or a product as described in, or in combination with, one or more of the preceding claims, wherein it is achieved to lower the number of B-cells, preferably anti-HIV- antibody producing B-cells, preferably anti-HIV coat- antibody producing B-cells.

41. A method and/or a product as described in-, or in combination with-, one or more of the preceding claims, wherein in vivo depletion is included, preferably with antibodies, preferably against HIV, preferably against the HIV envelope.

43. A method and/or a product as described in-, or in combination with-, one or more of the preceding claims, wherein in vivo depletion of antibodies is achieved for instance by other antibodies.

44. A method 3n(⁴h)r a product as described in-, or in combination with-, one or more of the precessing claims, wherein there is a use of bi-specific antibodies, preferably directed against the combination CD19 /CD3 and or CD20/CD3.

45. A method and or product as described in more of the preceding claims, wherein there is a use of B-cell apoptose induci substances, preferably APO-1.

46. A method and/or a product as described in-, or in combination with-, one or more of the preceding claims, wherein there is use of an other inhibition of B-cell antibody production preferably by TGF-beta.

47. A method and/or a product as described in, or in combination with-, one or more of the preceding claims, wherein activation of provirus of the HIV infected subject is performed, preferably by administration of growth factors, preferably cytokines, preferably IL-2.

48. A method and/or a product as described in. or in combination with-, one or more of the preceding claims, wherein passive immune therapy is included, preferably with immune globulin of HIV-uninfected subjects.

49 A method and/or a product as described in, or in combination with-, one or more of the preceding claims, wherein there is a use of a metalion, preferably Zinc, to obtain one or more of the effects and/or results and/or applications as described in one or more of the preceding claims.

50. Any therapy that contains one or more methods as described in one or more of the preceding claims.

51. The use of any preparation according to one or more of the preceding claims, that includes the stimulation of stem cell-replication.

52. The use of any preparation according to one or more of the preceding claims, in combination with other signal proteins and peptides.

53. Any conceivable combination of two or more of the preceding claims, either resulting or not resulting in synergistic activity.

54. A method for quantitative structure function analysis research of biologically active proteins or peptides selected from human receptors such as interleukins, haemopoietic growth factors, peptide hormones or protein hormones, signal peptides or signal proteins, for the introduction of one or more of the following features: enhanced biological activity, enhanced stability, suppressed antigenicity, acquired antagonistic activity or cell inhibitory activity, said method comprising applying a specific chemical modification of selected amino acids using
a) gradual chemical modification of the protein or peptide, followed by
b) monitoring the modification reaction with a mild and sensitive method such as non denaturing electrophoresis and/or electrospray mass spectrometry and said monitoring optionally further comprising confirming the overall structural integrity e.g. using Circular Dichroism Spectroscopy,
c) protease treatment,
d) mass spectrometry and
e) assaying biological activity of the modified product and optionally assaying stability of the modified product, said proteins or peptides preferably being selected from interleukins 1-8, interleukin 10, GM-CSF, TNF, insulin, prolactin and gamma IFN, more preferably GM-CSF, EPO or an interleukin being selected from interleukins 2-7 selected from the cytokine super family.

55. A method according to claim 1 wherein specific digestion with specific proteases and mass spectrometry is carried out for characterisation and localisation of the modified amino acids.

56. A method according to claim 1 or 2 wherein specific digestion with specific endoproteases and LDMS is carried out for characterisation and localisation of the modified amino acids, said endoprotease for example being Endo Glu C or Endo Lys C.

57. A method according to any of claims 1-3 wherein the modification is carried out by specific digestion with specific exoproteases and electrospray mass spectrometry is carried out for characterisation and localisation of the modified amino acids, suitably the exoprotease is N terminal e.g. Cathepsine C or C terminal e.g. carboxypeptidase Y.

58. A method according to any of claims 1-3, wherein the modification is chemical modification, said modification being alkylation and/or said modification being acylation, such as acetylation e.g. by lodo acetate or succinylation e.g. by succinic anhydride, said modification suitably being a modification under gradually varying conditions, wherein one or more of the following conditions are varied as follows: pH between 5.0 and 7.0, preferably in steps of 0.5 pH units, and/or time or reagent-concentrations are varied.

59. A method according to claim 5, wherein the modification is carried out in the presence of phosphate buffer, preferably in combination with acetic anhydride.

60. A method according to one or more of the preceding claims, for the introduction of an antagonistic and/or cell inhibitory activity, wherein the modification has specificity to one or more residues that are involved in catalytic activity e.g. wherein the modification is within or in close proximity to a partial or complete catalytic center, said modification preferably changing the catalytic activity, suitably said residue is a histidine residue.

61. A method according to any of the preceding claims, wherein the modification is within or in close proximity to a metal binding center, preferably a Zinc binding center, suitably said residue is a histidine residue.

62. A method according to one or more of the preceding claims, wherein the modification is performed by reversibly denaturing the substrate and adding chelating agent to remove the metal ion e.g. in the presence of urea and EDTA, said urea preferably having a concentration larger than 5 M and said EDTA preferably having a concentration of 50 mM.

63. A method according to one or more of the preceding claims, wherein the modification is specific for one type of amino acid, for instance an amine-residue and/or even is specific for only 1 amine-residue in the peptide or protein, said 1 amine for instance being the N-terminus.

64. A method according to any of the preceding claims wherein the substrate is human interleukin-3, said method preferably providing interleukin 3 modified only at one or more of the following residues: Ala¹, His²⁶, Lys²⁸, Lys⁶⁶, His⁹⁵, His⁹⁸, Lys¹⁰⁰, or Lys ¹¹⁶.

65. A method according to any of the preceding claims for the introduction of an antagonistic and/or cell inhibitory activity said method comprising disruption of phosphate binding.

66. A modified signal substance, preferably a protein hormone, peptide hormone, a growth factor, a heamopoietic growth factor, an interferon, an interleukin and/or a colony stimulating factor with an enhanced biological activity, antagonistic activity and/or cell inhibitory activity, wherein the modification is within or in close proximity to a partial or complete catalytic center, preferably such that the catalytic activity is changed, said modification further preferably being within or in close proximity to a metal binding center.

67. A modified signal substance being a Zinc binding signal peptide, preferably selected from Growth Hormone, prolactin and insulin, the same (cytokine) superfamily as the IL-3 receptor, preferably IL-2, IL-3, IL-4, IL-5, IL-6, IL-7, GM-CSF, Epo, IFN-gamma, more preferably selected from the following: IL-2, IL-3, IL-6, IFN-gamma, Growth Hormone, prolactin and insulin, said modified substance having an enhanced biological activity, antagonistic activity and/or cell inhibitory activity, wherein the modification is, preferably within or in close proximity to a Zinc binding center, such that the metal binding properties have been changed.

68. A substance according to claim 14, wherein the metal ion is within or in close proximity to a catalytic center, preferably said metal ion having a catalytic function in the unmodified substance.

69. A substance, as described in one or more of the preceding substance claims 13-15, wherein the modification for producing an antagonist is a chemical modification, preferably an alkylation, an acylation or molecular biological modification like a deletion mutation and/or a substitution mutation, most preferably the modification is an alkylation .

70. A substance, as described in one or more of the preceding substance claims 13-16, wherein the modification is of an amino acid involved in the binding of a metal ion, preferably a Histidine residue.

71. A substance, as described in one or more of the preceding substance claims 13-17, wherein the affinity of the signal substance for the receptor has not decreased by more than a factor 10, preferably has remained the same and more preferably has increased.

72. A substance according to any of the preceding substance claims, 13-18 wherein the concentration of substance required for significant inhibition is suitable for clinical application i.e. less than a hundred fold higher than the native substance concentration, said substance optionally further having increased receptor binding capacity.

73. A substance according to any of the preceding substance claims, 13-19 wherein the substance is interleukin 3 preferably human interleukin 3, most preferably modified only at one or more of the following residues: Ala¹, His²⁶, Lys²⁸, Lys⁶⁶, His⁹⁵, His⁹⁸, Lys¹⁰⁰, or Lys¹¹⁶ .

74. Substance according to claim 20, comprising at least one of the following characteristics - 0,1 ng of the substance, modified IL-3 inhibits almost 50% of 3ng/ml native IL-3 3ng/ml of the substance, modified IL-3 suppresses 80-90% thymidine incorporation of 30-100 ng/ml control IL-3 the substance modified IL-3 inhibits control IL-3 by a factor 10-100.

75. A substance, as described in one or more of the preceding substance claims 13-21, wherein the substance has acquired one of the following combinations of characteristics
a decreased stability and increased antagonistic activity for example acetylated IL-3,
a decreased stability and increased agonistic activity e.g. N-terminally proteased IL-3 e.g. Cathepsin C treated IL-3,
an increased stability and antagonistic activity e.g succinylated IL-3,
an increased stability in combination with an agonistic activity for example C-terminally proteased IL-3 e.g. Carboxypeptidase-Y treated IL-3.

76. Preparation for clinical application, containing a modified substance (both in mixed form and in chemically bound form) according to one or more of the preceding substance claims 13-22, optionally in combination with other signal proteins and peptides.

77. A method for preparing a substance according to any of claims 13-22 comprising carrying out the method steps as defined in any of claims 1-12.

78. A method of inhibition, suppression and/or cure of a HIV infection wherein the antibody levels are lowered by any of the following steps
suppression of antibody production by B-cells, suppression of generation and/or maturation of B-cells, preferably said B cells being anti-HIV- antibody producing B-cells, preferably anti-HIV coat—antibody producing B-cells,
plasmaphoresis, partial or complete plasma recovery or selective return of serum,
in vitro removal of antibodies, preferably HIV-reactive antibodies, preferably HIV-envelope reactive antibodies,
in vivo depletion, preferably with antibodies, preferably against HIV, preferably against the HIV envelope.
leukophoresis.

79. A method according to claim 25 comprising application of a preparation as described by claim 19 and/or application of a substance obtainable by a method according to any of the method claims 1-12.

80. A method according to claim 25 or 26, comprising application of bi-specific antibodies, preferably directed against the combination CD19 CD3 and or CD20 CD3.

81. A method according to any of the preceding method claims 25-27, comprising application of B-cell apoptose inducing substances, preferably APO-1 and/or application of TGF-β as inhibitor of B-cell antibody production.

82. A method for stumulating of stem cell-replication comprising application of a preparation according to claim 19 and/or a substance obtainable according to any of the method steps according to any of claims 1-12.

83. A method of gene therapy comprising applying a nucleic acid construct encoding a substance according to claims 13-22 to a subject to be treated, said therapy e.g. being directed at HIV infection.
